(19) United States
(12) Patent Application Publication
(10) Pub. No.: US 2025/0302350 A1
(43) Pub. Date: Oct. 2, 2025

(54) Blood Collection Device with Front-End Automation Features

(71) Applicant: Becton, Dickinson and Company, Franklin Lakes, NJ (US)

(72) Inventors: Kishore K. Bokka Srinivasa Rao, Lyndhurst, NJ (US); Charles Peter Althoff, West Nyack, NY (US)

(21) Appl. No.: 18/864,029

(22) PCT Filed: May 22, 2023
(86) PCT No.: PCT/US2023/023039

§ 371 Date: Nov. 8, 2024

(51) Int. Cl. A61B 5/15

(52) U.S. Cl. CPC A61B 5/150351; A61B 5/150022; A61B 5/150786

(57) ABSTRACT

A collection container for holding a blood sample, the collection container including a tube that defines a collection cavity, a lid provided on one end of the collection cavity, and a cover member for covering at least a portion of a side surface of the collection container near the lid, in which the cover member is configured to cover an outer circumferential surface of the collection container to create a uniform smooth surface around a circumference of the collection container.

### CROSS REFERENCE TO RELATED APPLICATION

This application claims the benefit of U.S. Provisional Patent Application No. 63/344,381, entitled “Blood Collection Device with Front-End Automation Features”, filed May 20, 2022, the entire disclosure of which is hereby incorporated by reference in its entirety.

### BACKGROUND OF THE INVENTION

### Field of the Invention

The present disclosure relates generally to a device for obtaining a biological sample. More particularly, the present disclosure relates to an integrated finger-based capillary blood collection device with the ability to lance and squeeze a finger, collect, stabilize, and dispense a blood sample in a controlled manner.

### Description of Related Art

Devices for obtaining and collecting biological samples, such as blood samples, are commonly used in the medical industry. One type of blood collection that is commonly done in the medical field is capillary blood collection, which is often done to collect blood samples for testing. Certain diseases, such as diabetes, require that a patient's blood be tested on a regular basis to monitor, for example, the patient's blood sugar levels. Additionally, test kits, such as cholesterol test kits, often require a blood sample for analysis. The blood collection procedure usually involves pricking a finger or other suitable body part in order to obtain the blood sample. Typically, the amount of blood needed for such tests is relatively small and a small puncture wound or incision normally provides a sufficient amount of blood for these tests. Various types of lancet devices have been developed, which are used for puncturing the skin of a patient to obtain a capillary blood sample from the patient.

Many different types of lancet devices are commercially available to hospitals, clinics, doctors' offices, and the like, as well as to individual consumers. Such devices typically include a sharp-pointed member, such as a needle, or a sharp-edged member, such as a blade, that is used to make a quick puncture wound or incision in the patient's skin in order to provide a small outflow of blood. In order to simplify capillary blood collection, lancet devices have evolved into automatic devices that puncture or cut the skin of the patient upon actuation of a triggering mechanism. In some devices, the needle or blade is kept in a standby position until it is triggered by the user. Upon triggering, the needle or blade punctures or cuts the skin of the patient, for example, on the finger. Often, a spring is incorporated into the device to provide the “automatic” force necessary to puncture or cut the skin of the patient.

One type of contact activated lancet device that features automatic ejection and retraction of the puncturing or cutting element from and into the device is U.S. Pat. No. 9,380,975, which is owned by Becton, Dickinson and Company, the assignee of the present application. This lancet device includes a housing and a lancet structure having a puncturing clement. The lancet structure is disposed within the housing and adapted for movement between a retaining or pre-actuated position wherein the puncturing element is retained within the housing, and a puncturing position, wherein the puncturing clement extends through a forward end of the housing. The lancet device includes a drive spring disposed within the housing for biasing the lancet structure toward the puncturing position, and a retaining hub retaining the lancet structure in the retracted position against the bias of the drive spring. The retaining hub includes a pivotal lever in interference engagement with the lancet structure. An actuator within the housing pivots the lever, thereby moving the lancet structure toward the rearward end of the housing to at least partially compress the drive spring, and releasing the lever from interference engagement with the lancet structure. The blood sample that is received is then collected and/or tested. This testing can be done by a Point-of-Care (POC) testing device or it can be collected and sent to a testing facility.

Use of lancet devices for capillary blood collection can be complex requiring a high skill level for the healthcare worker performing the blood collection procedure. The multi-step nature of the capillary blood collection process can introduce several variables that may cause sample quality issues, such as hemolysis, inadequate sample stabilization, and micro-clots. The use of lancet devices for obtaining blood samples can result in several variables that effect the collection of the capillary blood sample, including, but not limited to, holding the lancet still during the testing, obtaining sufficient blood flow from the puncture site, adequately collecting the blood, preventing clotting, and the like. Some of the most common sources of process variability are: (1) inadequate lancing site cleaning and first drop removal which can potentially result in a contaminated sample; (2) inconsistent lancing location and depth which could potentially result in insufficient sample volume and a large fraction of interstitial fluid; (3) inconsistent squeezing technique and excessive pressure near the lancing site to promote blood extraction (e.g., blood milking) which could potentially result in a hemolyzed sample; (4) variable transfer interfaces and collection technique which could potentially result in a hemolyzed or contaminated sample; and (5) inadequate sample mixing with an anticoagulant which could potentially result in micro-clots.

During assembly and/or use of the blood collection devices, automated arms may be used to pick up and place the blood collection devices in desired locations. Due to features on the blood collection devices, including a protruding latch on the cap, the protruding living hinge on the cap, and/or the protruding U-shaped ribs for de-capping the cap, can create issues with the pick-and-place automated arms. Therefore, there is a current need in the art for a blood collection device that includes front-end features to improve automated movement and securement of the blood collection device to a pair of automated arms.

### SUMMARY OF THE INVENTION

According to one aspect of the present disclosure, a collection container for holding a blood sample may include a tube having an open end, a closed bottom end, and a sidewall extending therebetween that defines a collection cavity, the sidewall defining an exterior side surface; a lid removably engageable with the open end of the tube; and a cover member for covering at least a portion of the exterior side surface of the tube proximate the lid, wherein the cover member is configured to circumferentially cover at least a portion of the exterior side surface of the collection container to create a uniform smooth surface around a circumference of the collection container.

According to one aspect of the present disclosure, the lid may include a release tab that releases the collection container from a blood collector attachment when pressed. The lid may be connected to the collection container via a living hinge. The cover member may be integrally formed with the collection container. The cover member may include a colorant to identify the cover member. The colorant may be yellow colored. A diameter of the lid may be greater than a diameter of the tube. An extended portion may be removably attached to a bottom end of the tube.

According to one aspect of the present disclosure, a collection container for holding a blood sample may include a tube having an open end, a closed bottom end, and a sidewall extending therebetween that defines a collection cavity, the sidewall defining an exterior side surface; a lid removably engageable with the open end of the tube; and a plurality of axial ribs configured to cover at least a portion of the exterior side surface of the tube proximate the lid, wherein the plurality of axial ribs are configured to circumferentially cover at least a portion of the exterior side surface to create a more uniform banded smooth surface around a circumference of the collection container.

According to one aspect of the present disclosure, the lid may include a release tab that releases the collection container from a blood collector attachment when pressed. The lid may be connected to the collection container via a living hinge. The plurality of axial ribs may be integrally formed with the collection container. The plurality of axial ribs may include a colorant to identify the plurality of axial ribs. The colorant may be yellow colored. A diameter of the lid may be greater than a diameter of the tube. An extended portion may be removably attached to a bottom end of the tube.

According to one aspect of the present disclosure, a collection container for holding a blood sample may include a tube having an open end, a closed bottom end, and a sidewall extending therebetween that defines a collection cavity, the sidewall defining an exterior side surface; a lid removably engageable with the open end of the tube; and an extended portion removably engageable with a portion of the tube, such that the extended portion extends over the closed bottom end and is configured to increase an overall length of the tube. The extended portion may be removably engageable with the tube via a friction fit, an annular snap fit connection, a threaded connection, a bayonet connection, or an ultrasonic welding connection.

According to one aspect of the present disclosure, a collection container for holding a blood sample may include a tube having an open end, a closed bottom end, and a sidewall extending therebetween that defines a collection cavity, the sidewall defining an exterior side surface; a lid removably engageable with the open end of the tube; a cover member for covering at least a portion of the exterior side surface of the tube proximate the lid; and a plurality of ribs positioned on the cover member and configured to cover at least a portion of the cover, wherein the cover and the plurality of ribs are configured to circumferentially cover at least a portion of the exterior side surface to create a more uniform banded smooth surface around a circumference of the collection container.

The invention is also further defined in the following clauses:

Clause 1: A collection container for holding a blood sample, the collection container comprising: a tube having an open end, a closed bottom end, and a sidewall extending therebetween that defines a collection cavity, the sidewall defining an exterior side surface; a lid removably engageable with the open end of the tube; and a cover member for covering at least a portion of the exterior side surface of the tube proximate the lid, wherein the cover member is configured to circumferentially cover at least a portion of the exterior side surface of the collection container to create a uniform smooth surface around a circumference of the collection container.

Clause 2: The collection container of clause 1, wherein the lid includes a release tab that releases the collection container from a blood collector attachment when pressed.

Clause 3: The collection container of clause 1 or clause 2, wherein the lid is connected to the collection container via a living hinge.

Clause 4: The collection container of any of clauses 1-3, wherein the cover member is integrally formed with the collection container.

Clause 5: The collection container of any of clauses 1-4, wherein the cover member includes a colorant to identify the cover member.

Clause 6: The collection container of clause 5, wherein the colorant is yellow colored.

Clause 7: The collection container of any of clauses 1-6, wherein a diameter of the lid is greater than a diameter of the tube.

Clause 8: The collection container of any of clauses 1-7, further comprising an extended portion removably attached to a bottom end of the tube.

Clause 9: A collection container for holding a blood sample, the collection container comprising: a tube having an open end, a closed bottom end, and a sidewall extending therebetween that defines a collection cavity, the sidewall defining an exterior side surface; a lid removably engageable with the open end of the tube; and a plurality of axial ribs configured to cover at least a portion of the exterior side surface of the tube proximate the lid, wherein the plurality of axial ribs are configured to circumferentially cover at least a portion of the exterior side surface to create a more uniform banded smooth surface around a circumference of the collection container.

Clause 10: The collection container of clause 9, wherein the lid includes a release tab that releases the collection container from a blood collector attachment when pressed.

Clause 11: The collection container of clause 9 or clause 10, wherein the lid is connected to the collection container via a living hinge.

Clause 12: The collection container of any of clauses 9-11, wherein the plurality of axial ribs is integrally formed with the collection container.

Clause 13: The collection container of any of clauses 9-12, wherein the plurality of axial ribs includes a colorant to identify the plurality of axial ribs.

Clause 14: The collection container of clause 13, wherein the colorant is yellow colored.

Clause 15: The collection container of any of clauses 9-14, wherein a diameter of the lid is greater than a diameter of the tube.

Clause 16: The collection container of any of clauses 9-15, further comprising an extended portion removably attached to a bottom end of the tube.

Clause 17: A collection container for holding a blood sample, the collection container comprising: a tube having an open end, a closed bottom end, and a sidewall extending therebetween that defines a collection cavity, the sidewall defining an exterior side surface; a lid removably engageable with the open end of the tube; and an extended portion removably engageable with a portion of the tube, such that the extended portion extends over the closed bottom end and is configured to increase an overall length of the tube.

Clause 18: The collection container of clause 17, wherein the extended portion is removably engageable with the tube via a friction fit, an annular snap fit connection, a threaded connection, a bayonet connection, or an ultrasonic welding connection.

Clause 19: A collection container for holding a blood sample, the collection container comprising a tube having an open end, a closed bottom end, and a sidewall extending therebetween that defines a collection cavity, the sidewall defining an exterior side surface; a lid removably engageable with the open end of the tube; a cover member for covering at least a portion of the exterior side surface of the tube proximate the lid; and a plurality of ribs positioned on the cover member and configured to cover at least a portion of the cover, wherein the cover and the plurality of ribs are configured to circumferentially cover at least a portion of the exterior side surface to create a more uniform banded smooth surface around a circumference of the collection container.

### BRIEF DESCRIPTION OF THE DRAWINGS

FIG. 1 is a perspective view of a holder in accordance with an embodiment of the present invention.

FIG. 2A is a cross-sectional view of a device for obtaining a blood sample from a patient's finger and a lancet in accordance with another embodiment of the present disclosure.

FIG. 2B is a perspective view of a device for obtaining a blood sample from a patient's finger and a sample collection container in accordance with another embodiment of the present disclosure.

FIG. 3 is a side view of a device for obtaining a blood sample from a patient's finger and a collection container in accordance with another embodiment of the present disclosure.

FIG. 4 is a perspective view of a collection container detachment member being unlocked.

FIG. 5 is a perspective view of the collection container of FIG. 4 in a detached state.

FIG. 6 is another perspective view of the collection container of FIG. 4 in the detached state.

FIG. 7 is a perspective view of a collection container with a cover according to one aspect of the present disclosure.

FIG. 8 is a perspective view of a collection container with ribs according to one aspect of the present disclosure.

FIG. 9A is a perspective view of the collection container and cover of FIG. 7 with an added colorant.

FIG. 9B is a perspective view of the collection container and cover of FIG. 7 with an added colorant.

FIG. 10A is a perspective view of the collection container and ribs of FIG. 8 with an added colorant.

FIG. 10B is a perspective view of the collection container and ribs of FIG. 8 with an added colorant.

FIG. 11 is a perspective view of a collection container with an extended portion according to one aspect of the present disclosure.

FIG. 12 is a cross-sectional view of the collection container of FIG. 11.

FIG. 13 is a cross-section view of a collection container according to one aspect of the present disclosure in which the cap has a larger diameter than a tube of the collection container.

FIG. 14 is an assembly view of a collection container with ribs and an extended portion according to one aspect of the present disclosure.

### DESCRIPTION OF THE DISCLOSURE

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

The present disclosure is directed to a device for obtaining a biological sample, such as a capillary blood collection device, which meets the needs set forth above and has the ability to lance and squeeze the finger, collect the sample, stabilize the sample, and subsequently dispense the sample in a controlled manner. The device also simplifies and streamlines the capillary blood collection by eliminating workflow variabilities which are typically associated with low sample quality including hemolysis and micro-clots.

Blood collection is fundamentally driven by pressure-driven flow. Devices or techniques either reduce the pressure outside the blood vessel (vacuum-powered flow) or increase the pressure inside the vessels. Both approaches increase the difference between the blood vessel pressure and external pressure, and increase the flow rate from inside the vessel to outside where the collection container is present. The location of squeezing can also be critical, as soft tissues (e.g. fat, skin, and musculature) are perfused with blood while hard tissues and joints are poorly perfused or are too mechanically stable to compress without patient pain.

Red blood cells (RBCs) are subject to hemolysis during collection. Hemolysis (RBC destruction) contaminates samples for diagnostic analysis, both by spilling cell contents into the liquid serum of the sample and by coloring the serum red via hemoglobin and interfering with colorimetric reactions. The amount of hemolysis during collection is driven by shear-mediated destruction of the cells due to flow rate and flow path as well as pressure-driven hemolysis where physical compression of tissues and vessels can damage cells. Hemolysis can therefore be controlled by ensuring that applied pressures and flows are not too high in any of the locations of the finger being squeezed.

The present disclosure includes a self-contained and fully integrated finger-based capillary blood collection device with ability to lance, collect, and stabilize high volume capillary blood samples, e.g., up to or above 500 microliters. The device simplifies and streamlines high volume capillary blood collection by eliminating workflow steps and variabilities which are typically associated with low sample quality including hemolysis, micro-clots, and patient discomfort. The device comprises a retractable lancing mechanism that can lance the finger and an associated blood flow path which ensures attachment and transfer of the capillary blood from the pricked finger site to the collection container. The device also includes a holder that can be cyclically squeezed to stimulate, i.e., pump, blood flow out of the finger and also an anticoagulant deposited in the flow path or collection container to stabilize collected sample.

According to one design, the device can comprise discrete components such as a holder, a lancet, and a collection container. According to another design, the lancet and collection container can be integrated into one device which is then used with the holder. According to yet another design, the holder, lancet, and collection container can be integrated into a single system. Any of these designs are envisioned to be used as a self-standing disposable device and/or in association with an external power source for pain reduction control. The capillary blood collection device can serve as a platform for various capillary collection containers ranging from small tubes to capillary dispensers, as well as on-board plasma separation modules. This capability extends the product flexibility to various applications including dispensing to a Point-of-Care (POC) cartridge or to a small collection tube transfer which can be used in a centrifuge or an analytical instrument.

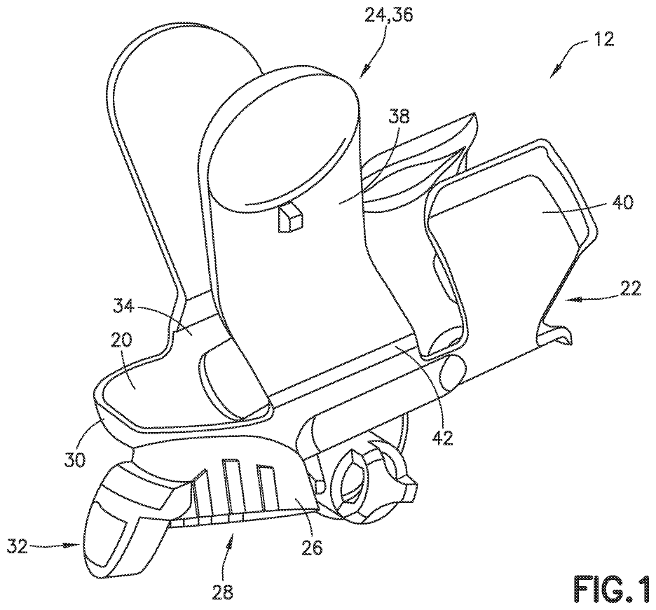

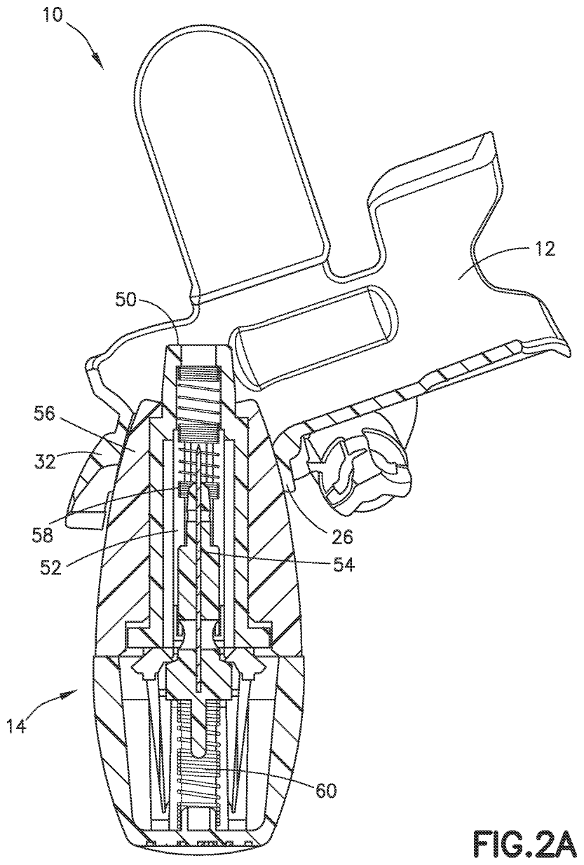

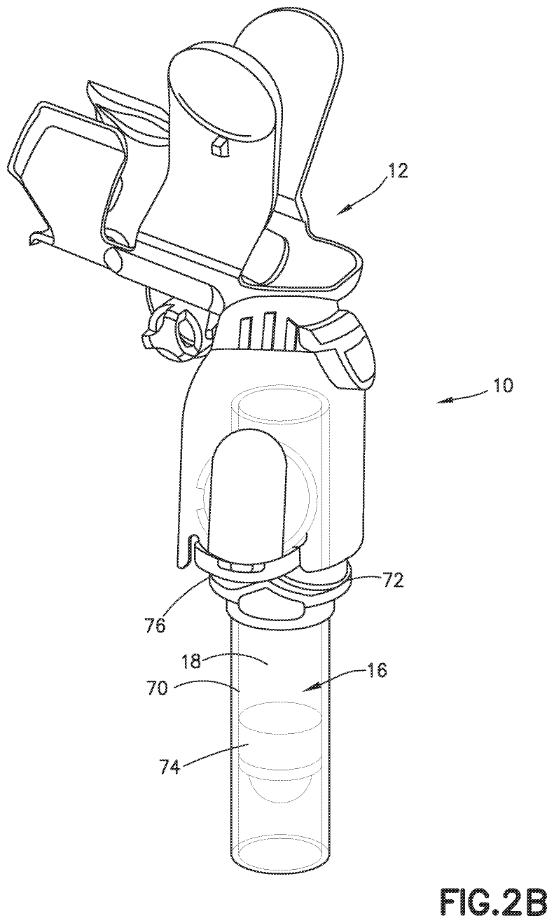

Referring to FIGS. 1 and 2A, in an exemplary embodiment, a device 10 of the present disclosure includes discrete components, e.g., a holder 12 (as shown in FIG. 1), a lancet housing or lancet 14 (as shown in FIG. 2A), and a collection container 16 (as shown in FIG. 2B). In another exemplary embodiment, a semi-integrated device of the present disclosure may include an at-angle flow and include an integrated lancet housing and collection container which can be connected with a separate holder. In another exemplary embodiment, a semi-integrated device of the present disclosure may have an in-line flow and include an integrated lancet housing and collection container which can be connected with a separate holder. In another exemplary embodiment, an integrated device of the present disclosure may have an at-angle flow and include an integrated holder, lancet housing, and collection container. In another exemplary embodiment, an integrated device of the present disclosure may have an in-line flow and include an integrated holder, lancet housing, and collection container.

Referring to FIG. 1, an exemplary embodiment of a holder 12 of the present disclosure that is able to receive a sample source, e.g., a finger 19, for supplying a biological sample, such as a blood sample 18, is shown and described. A holder 12 of the present disclosure generally includes a finger receiving portion 20 having a first opening 22 (FIG. 1), an actuation portion 24, a port 26 having a second opening 28, and a finger end guard 30. In one embodiment, the finger end guard 30 provides a stop portion for properly aligning and securing a finger 19 within the holder 12. The finger end guard 30 further assists in ensuring the patient's finger 19 is placed at a proper position within the finger receiving portion 20 so that applied pressure to the patient's finger 19 will result in adequate blood flow.

The first opening 22 of the finger receiving portion 20 is configured for receiving a sample source, e.g., a finger 19, for supplying a biological sample, such as a blood sample 18. It can be appreciated that the sample source could include other parts of the body capable of fitting within the first opening 22. The port 26 is in communication with the finger receiving portion 20. For example, with a finger 19 received within the holder 12, the port 26 is in communication with a portion of the finger 19. A holder 12 of the present disclosure can be sized to accommodate all finger sizes.

The second opening 28 of the port 26 is configured for receiving a lancet housing 14 and a collection container 16 as described in more detail below. In one embodiment, the port 26 includes a locking portion 32 for securely receiving the lancet housing 14 and the collection container 16 within the port 26.

In one embodiment, the actuation portion 24 is transitionable between a first position in which the holder 12 defines a first diameter and a second position which the holder 12 defines a second diameter, wherein the second diameter is less than the first diameter. In one embodiment, the actuation portion 24 is transitionable between a first position in which the holder 12 defines a first elliptical shape, and a second position in which the holder 12 defines a second elliptical shape, wherein the first elliptical shape is different than the second elliptical shape. In this manner, with the holder 12 in the second position with a reduced diameter, a portion of the holder 12 contacts the sample source and the actuation portion 24 of the holder 12 is able to pump and/or extract blood 18 as described in more detail below.

Referring to FIG. 1, in one embodiment, the actuation portion 24 includes a contact member 34. With the actuation portion 24 in the first position, the contact member 34 is in a disengaged position, i.e., the contact member 34 is provided in a first position with respect to a sample source, e.g., the finger 19, such that the contact member 34 may be in slight contact therewith. With the actuation portion 24 in the second position, the contact member 34 is in an engaged position, i.e., the contact member 34 is provided in a second position with respect to the sample source, e.g., the finger 19, such that the contact member 34 is in an applied pressure contact with the finger 19, and the actuation portion 24 of the holder 12 is able to pump and/or extract blood 18. For example, with the contact member 34 in the engaged position, the contact member 34 exerts a pressure on the sample source.

Referring to FIG. 1, in one embodiment, the actuation portion 24 includes a pumping member 36 for applying pressure to the sample source, e.g., the finger 19. In one embodiment, the pumping member 36 comprises a pair of opposed tabs or wings 38. In such an embodiment, each tab 38 may include a contact member 34. In one embodiment, the holder 12 includes a living hinge portion 42. The living hinge portion 42 allows a user to squeeze the wings 38 between a first position (passive state) and a second position (active state). The use of the tabs or wings 38 to draw blood 18 out of a patient's finger 19 minimizes hemolysis while maintaining an adequate flow of blood from the patient's finger 19. A resting position and hinge of the wings 38 are designed to maintain contact and retention with the smallest patient finger that can fit into a holder 12 while flexing to accommodate the largest patient finger within a holder 12 without blood occlusion.

Advantageously, the holder 12 of the present disclosure allows a user to repeatedly squeeze and release the wings 38 to pump and/or extract blood 18 from a finger 19 until a desired amount of blood 18 is filled in a collection container 16. The wings 38 are configured to flex to maintain gentle contact with a range of patient finger sizes that may be used with the holder 12 and to retain the holder 12 on the patient's finger 19.

Advantageously, with the holder 12 placed onto a finger 19, the holder 12 does not constrict the blood flow and defines lancing and finger squeezing locations. The squeezing tabs or wings 38 provide a pre-defined range of squeezing pressure that is consistently applied throughout a finger 19. By doing so, the holder 12 provides a gentle controlled finger massage that stimulates blood extraction and minimizes any potential hemolysis.

Referring to FIG. 1, in one embodiment, the holder 12 includes a stability extension portion 40. This provides additional support for the holder 12 to be securely placed onto a finger 19. In one embodiment, the finger receiving portion 20 forms a generally C-shaped member and includes a plurality of inner gripping members for providing additional grip and support for the holder 12 to be securely placed onto a finger 19. The stability extension portion 40 assists in maintaining contact with the patient's finger 19 during use of the holder 12 while avoiding the blood supply and knuckles of the patient's finger 19.

In one embodiment, the finger receiving portion 20 is formed of a flexible material. In some embodiments, the finger receiving portion 20 and the port 26 are formed from a flexible material.

A device 10 for obtaining a blood sample 18 of the present disclosure includes a lancet housing or lancet 14 that is removably connectable to a port 26 of a holder 12. In one embodiment, the lancet housing 14 includes an inlet or opening 50, an interior 52, a puncturing clement 54, an engagement portion 56, a retractable mechanism 58, and a drive spring 60. In one embodiment, the puncturing element 54 is moveable between a pre-actuated position wherein the puncturing element 54 is retained within the interior 52 of the lancet housing 14 and a puncturing position wherein at least a portion of the puncturing element 54 extends through the inlet 50 of the lancet housing 14 to lance a portion of a finger 19.

In one embodiment, the lancet 14 of the present disclosure is a contact activated lancet and may be constructed in accordance with the features disclosed in U.S. Patent Application Publication No. 2006/0052809 filed May 6, 2005, entitled “Contact Activated Lancet Device”, and commonly assigned with the present application, the entire disclosure of which is hereby expressly incorporated herein by reference thereto.

In one embodiment, the lancet housing 14 may be a separate component from the holder 12 and the collection container 16. In some embodiments, the collection container 16 and the lancet housing 14 form a single component that is removably connectable to the port 26 of the holder 12. In some embodiments, the collection container 16, the lancet housing 14, and the holder 12 form a single component.

Referring to FIG. 2A, in one embodiment, with the holder 12 and the lancet housing 14 being separate components, the lancet housing 14 is removably connectable to the port 26 of the holder 12. In such an embodiment, the lancet housing 14 includes an engagement portion 56. Referring to FIG. 2A, in one embodiment, the lancet housing 14 is pushed into the port 26 of the holder 12 such that the engagement portion 56 of the lancet housing 14 is locked within the locking portion 32 of the holder 12. In this manner, the lancet housing 14 is securely connected and locked to the holder 12 such that the puncturing element 54 of the lancet housing 14 can be activated to lance or puncture a sample source, e.g., a finger 19. In some embodiments, the port 26 of the holder 12 includes a plurality of ribs for securing and locking the lancet 14 or the collection container 16 in the port 26.

To activate the lancet 14, the lancet 14 is pushed against a finger 19 to activate a retractable mechanism 58 of the lancet 14 to lance a finger 19. The lancet 14 of the present disclosure consistently delivers correct lancing depth and a pre-defined lancing location, thus ensuring a sufficient sample volume.

In one embodiment, the lancet 14 includes a drive spring 60 disposed within the interior 52 of the lancet housing 14 for biasing the puncturing element 54 toward the puncturing position. After puncturing, the puncturing clement 54 is immediately retracted and safely secured within the interior 52 of the lancet housing 14.

In one embodiment, the lancet 14 of the present disclosure is used to lance the skin of a finger 19 and then a blood sample 18 is squeezed into a collection container 16 as described in more detail below.

In one embodiment, the lancet housing 14 of the present disclosure is used to lance the skin of a finger 19 along a lance path and then a blood sample 18 flows down a blood flow path at an angle to the lance path as described in more detail below.

In one embodiment, the lancet 14 can include a hollow needle. In such an embodiment, the lancet housing 14 of the present disclosure is used to lance the skin of a finger 19 along a lance path and then a blood sample 18 (shown in FIG. 2B) flows along a parallel blood flow path through the hollow needle.

As shown in FIG. 2B, a device 10 for obtaining a blood sample 18 of the present disclosure includes a collection container 16 that is removably connectable to the port 26 of the holder 12. In one configuration, the collection container 16 may be any size collection container, such as a microtube that is adaptable to fit within the profile of a 13×75 mm, 13×100 mm, or 16×100 mm standard tube collection container. The collection container 16 defines a collection cavity 70 for receiving a blood sample 18, a container engagement portion 72, a blood collector portion 74, and a cap or septum 76. Once a desired amount of blood 18 is collected within the container 16, a blood collector portion 74 is detached from the collection device 10 in order to send a collected sample 18 to a diagnostic instrument and/or testing device. The blood collector portion 74 is sealed via the cap or septum 76 once removed from the collection device 10 to protectively seal the blood sample 18 within the collection cavity 70.

In one embodiment, the collection container 16 may be a separate component from the holder 12 and the lancet housing 14. In some embodiments, the collection container 16 and the lancet housing 14 form a single component that is removably connectable to the port 26 of the holder 12. In some embodiments, the collection container 16, the lancet housing 14, and the holder 12 form a single component.

In one embodiment, with the holder 12 and the collection container 16 being separate components, the container 16 is removably connectable to the port 26 of the holder 12. In such an embodiment, the container 16 includes a container engagement portion 72. In one embodiment, the container 16 is pushed into the port 26 of the holder 12 such that the container engagement portion 72 of the container 16 is locked within the locking portion 32 of the holder 12. In this manner, the container 16 is securely connected and locked to the holder 12 such that a blood sample 18 can safely flow from the finger 19 within the holder 12 to the collection cavity 70 of the container 16.

It can be appreciated that several types of collection containers 16 can be used with the device 10 of the present disclosure. It can also be appreciated that the collection container 16 can be associated with a separate dispensing unit or the collection container 16 can include an integral dispensing portion for dispensing the blood 18 to a testing device. The collection container 16 could be a stand-alone container, or could also include a false bottom to allow instrument compatibility with conventional larger volume collection containers. Optionally, the bottom of the collection container 16 may be hemispherical or include a partially rounded portion.

Referring to FIG. 1, use of a device 10 of the present disclosure having discrete components, e.g., a holder 12, a lancet housing or lancet 14, and a collection container 16, will now be described.

Referring to FIG. 1, first a desired finger 19 is cleaned and a holder 12 having an appropriate size for the desired finger 19 is selected and placed onto the finger 19 securely. Next, referring to FIG. 2A, a lancet housing 14 is connected to the port 26 of the holder 12. As discussed above, the lancet housing 14 is pushed into the port 26 of the holder 12 such that the engagement portion 56 of the lancet housing 14 is locked within the locking portion 32 of the holder 12. In this manner, the lancet housing 14 is securely connected and locked to the holder 12 such that the puncturing element 54 (FIG. 2A) of the lancet housing 14 can be activated to lance or puncture a sample source, e.g., a finger 19. With the lancet 14 connected to the port 26 of the holder 12, the lancet 14 is in communication with the finger 19.

When it is desired to activate the lancet 14 to lance the skin of a finger 19, the lancet 14 is pushed against a finger 19 to activate a retractable mechanism 58 (FIG. 2A) of the lancet 14 to lance a finger 19. The lancet 14 of the present disclosure consistently delivers correct lancing depth and a pre-defined lancing location, thus ensuring a sufficient sample volume.

After the finger 19 is lanced to create blood 18 flow from the finger 19, the lancet 14 is removed from the holder 12 and the collection container 16 is pushed into the port 26 of the holder 12. Referring to FIG. 2B, the container 16 is pushed into the port 26 of the holder 12 such that the container engagement portion 72 of the container 16 is locked within the locking portion 32 of the holder 12. In this manner, the container 16 is securely connected and locked to the holder 12 such that a blood sample 18 can safely flow from the finger 19 within the holder 12 to the collection cavity 70 of the container 16.

Referring to FIG. 1, with the container 16 properly secured to the holder 12 for collection of a blood sample 18, a user is able to repeatedly squeeze and release the wings 38 of the holder 12 to pump and/or extract blood 18 from a finger 19 until a desired amount of blood 18 is filled in a collection container 16. Advantageously, with the holder 12 placed onto a finger 19, the holder 12 does not constrict the blood flow and defines lancing and finger squeezing locations. The squeezing tabs or wings 38 provide a pre-defined range of squeezing pressure that is consistently applied throughout a finger 19. By doing so, the holder 12 provides a gentle controlled finger 19 massage that stimulates blood extraction and minimizes any potential hemolysis.

For example, referring to FIG. 1, in one embodiment, the actuation portion 24 includes a contact member 34. With the actuation portion 24 in the first position, the contact member 34 is in a disengaged position, i.e., the contact member 34 is in the first position with respect to the sample source, e.g., the finger 19. With the actuation portion 24 in the second position, the contact member 34 is in an engaged position, i.e., the contact member 34 is in the second position and in applied pressure contact with a sample source, e.g., the finger 19, and the actuation portion 24 of the holder 12 is able to pump and/or extract blood 18. For example, with the contact member 34 in the engaged position, the contact member 34 exerts a pressure on the sample source.

Once a desired amount of blood 18 is collected within the container 16, a blood collector portion 74 is detached from the collection device 10 in order to send a collected sample 18 to a diagnostic instrument and/or testing device. The blood collector portion 74 is scaled via the cap or septum 76 once removed from the collection device 10 to protectively seal the blood sample 18 within the collection cavity 70.

The devices of the present disclosure are compatible with any known testing device, whether the testing device is off-site or a point-of-care testing device. Various point-of-care testing devices are known in the art. Such point-of-care testing devices include test strips, glass slides, diagnostic cartridges, or other testing devices for testing and analysis. Test strips, glass slides, and diagnostic cartridges are point-of-care testing devices that receive a blood sample and test that blood for one or more physiological and biochemical states. There are many point-of-care devices that use cartridge based architecture to analyze very small amounts of blood bedside without the need to send the sample to a lab for analysis. This saves time in getting results over the long run, but creates a different set of challenges versus the highly routine lab environment. Examples of such testing cartridges include the i-STAT® testing cartridge from the Abbot group of companies. Testing cartridges such as the i-STAT® cartridges may be used to test for a variety of conditions including the presence of chemicals and electrolytes, hematology, blood gas concentrations, coagulation, or cardiac markers. The results of tests using such cartridges are quickly provided to the clinician.

The collection container 16 may also contain a sample stabilizer, e.g., an anticoagulant, to stabilize a blood sample 18 and/or a component of a blood sample 18 disposed therein. The collection container 16 may also include at least one fill line(s) corresponding to a predetermined volume of sample. The collection container may also indicate/meter a collected volume of blood.

Any of the devices for obtaining a blood sample of the present disclosure can be used as a self-standing disposable device and/or in association with an external power source for pain reduction control. For example, a portion of holder 12 may include embedded electrodes which receive a signal from an external pain control module to deliver at least one of heat, vibration, or transcutaneous electrical nerve stimulation (TENS) for pain reduction control. The devices for obtaining a blood sample of the present disclosure may also include various options for on-board plasma separation. The devices for obtaining a blood sample of the present disclosure may also include a unique sample identifier that can be paired with patient information at the time of collection. The devices for obtaining a blood sample of the present disclosure may also include on-board diagnostic feedback at the time of collection. A device for obtaining a blood sample of the present disclosure may also allow for dual collection, e.g., the collection of two samples into two separate containers, using multiple collection ports which enable the collection of multiple samples from the same source and treating the samples with different sample stabilizers, such as anticoagulants.

A device for obtaining a blood sample of the present disclosure significantly simplifies and de-skills large volume capillary collection from a finger relative to the conventional capillary collection using lancet and capillary tube. The devices of the present disclosure eliminate blood exposure and prevents device reuse.

The devices for obtaining a blood sample of the present disclosure simplify, deskill, and streamline the collection process. This is all achieved by a self-contained closed system device which after it is placed onto a finger will provide lancing, blood extraction, stabilization, and containment functions, all in one unit.

The devices for obtaining a blood sample of the present disclosure may be associated with a self-standing unit that provides automated pumping, controlled finger squeezing, and automated sample labeling and processing.

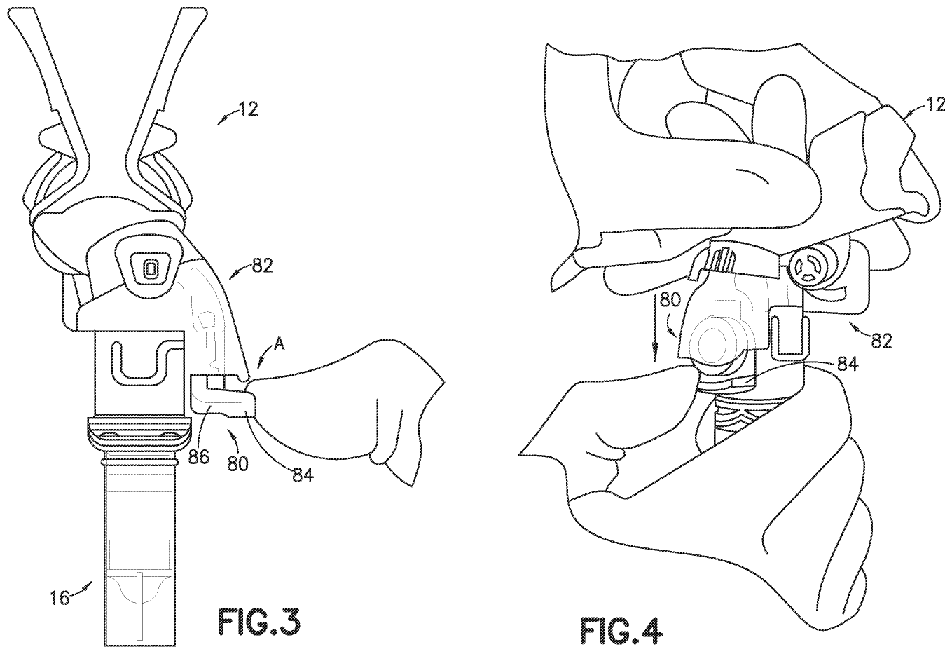

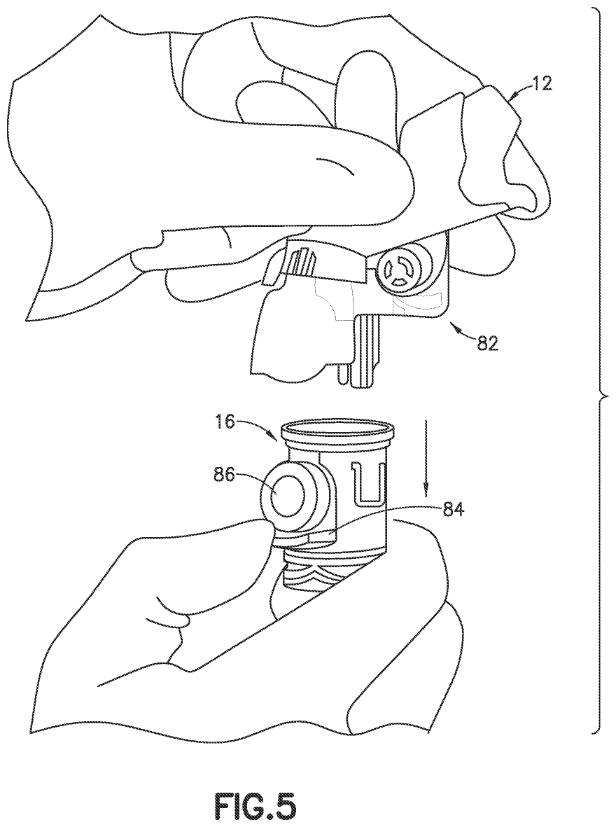

With reference to FIGS. 3-5, according to one embodiment of the present disclosure, a collection container detachment member 80 is shown and described in detail. The collection container detachment member 80 may be an intuitive release mechanism that permits a user to press release the member 80 to release the collection container 16 from a blood collector attachment 82 removably connected to the holder 12. The collection container detachment member 80 provides haptic feedback to the user to indicate that the collection container 16 has been removed from the blood collector attachment 82.

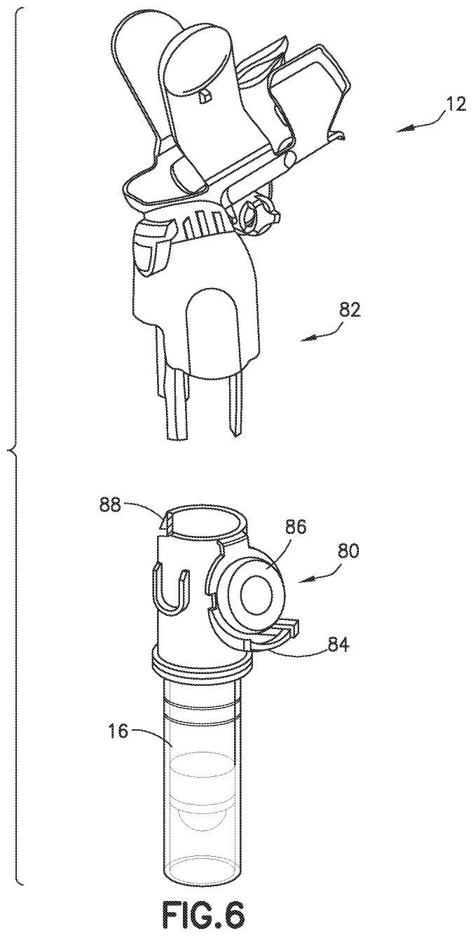

With reference to FIG. 6, according to one embodiment of the present disclosure, the collection container detachment member 80 may include a release tab 84 that permits a user to effect a disconnection of the collection container 16 from the blood collector attachment 82. In one embodiment, the release tab 84 may be pressed inwardly by the user to release the collection container 16 from the blood collector attachment 82 after the blood sample 18 has been drawn from the patient. Once the release tab 84 has been pressed by the user, the collection container 16 may be pulled away from the blood collector attachment 82. The release tab 84 may include a latch feature that prevents contamination to the surfaces that contact the blood sample 18 in the collection container 16. Since the release tab 84 must be positively engaged by the user to release the collection container 16, the release tab 84 prevents any accidental release of the collection container 16 from the blood collector attachment 82. In one embodiment, the release tab 84 may have a bright color or pattern that visually identifies to the user that the release tab 84 must be pressed to release the collection container 16 from the blood collector attachment 82. In one embodiment, the release tab 84 acts as a “lever” for unlocking the collection container detachment member 80. In one embodiment of the present disclosure, the blood collector attachment 82 may also shield the sealing surface of the lid 86 from contamination until the collection container 16 is removed and the lid 86 is closed on the collection container 16. In one embodiment of the present disclosure, the latching feature of the lid 86 may be blocked by the blood collector attachment 82 to provide a tamper-proof feature that identifies tampering of the lid 86.

With reference to FIG. 7, according to one non-limiting aspect or example of the present disclosure, the blood collection device 10 may also include features that assist in securing or picking up the blood collection device 10 by an automated machine or device. Due to several features of the blood collection device 10 creating obstacles or hindrances to arms of an automated pick-up machine that attempt to pick up the blood collection device 10, a cover member, such as cover 100 may be provided around an outer circumference of the collection container 16 to create a uniform radial surface for gripping and/or holding the collection container 16 during pick-and-place automation. The cover 100 may extend around the entire outer circumference of a top portion of the collection container 16. In one example, the cover 100 may be an over-mold of a rigid plastic or a semi-rigid elastomeric material. The cover 100 is configured to improve the ability of the arms or grippers of an automated machine or device to grip the collection container 16 for transport or relocation. The cover 100 may be formed as a single monolithic piece or as a plurality of pieces that are connected to one another. The cover may be opaque in color to assist in tube recognition. The cover may also provide uniform axial symmetry about a circumferential perimeter of the exterior surface of the at least a portion of the collection container 16, such as adjacent the open end.

With reference to FIG. 8, according to one non-limiting aspect or example of the present disclosure, the blood collection device 10 may include an alternative feature that assists in securing or picking up the blood collection device 10 by an automated machine or device. Instead of the cover 100 described above, a plurality of axial ribs 102 may be positioned around the circumference of the top portion of the collection container 16 to create a uniform radial surface for gripping and/or holding the collection container 16 during pick-and-place automation. The plurality of axial ribs 102 may extend around the entire outer circumference of the collection container 16. A longitudinal axis of each axial rib 102 may extend parallel to a longitudinal axis of the collection container 16. The axial ribs 102 ensure that there are not one or two protruding edges on the collection container 16 but, instead, includes a more uniform smooth surface. The axial ribs 102 are configured to fill in the negative space below the various protrusions on the collection container 16, including the negative space below the lid latch and the living hinge. The axial ribs 102 may be made of a rigid plastic or a semi-rigid elastomeric material. The axial ribs 102 may be provided on the collection container 16 during primary molding of the collection container 16 or as an over-molded feature on the collection container 16. In another example, instead of the axial ribs 102, a plurality of rings could be positioned along the length of the top portion of the collection container 16 to also create a more uniform smooth surface that can more easily be gripped by arms or grippers of an automated pick-and-place machine or device. The rings could be radial rings that extend around the outer circumference of the top portion of the collection container 16.

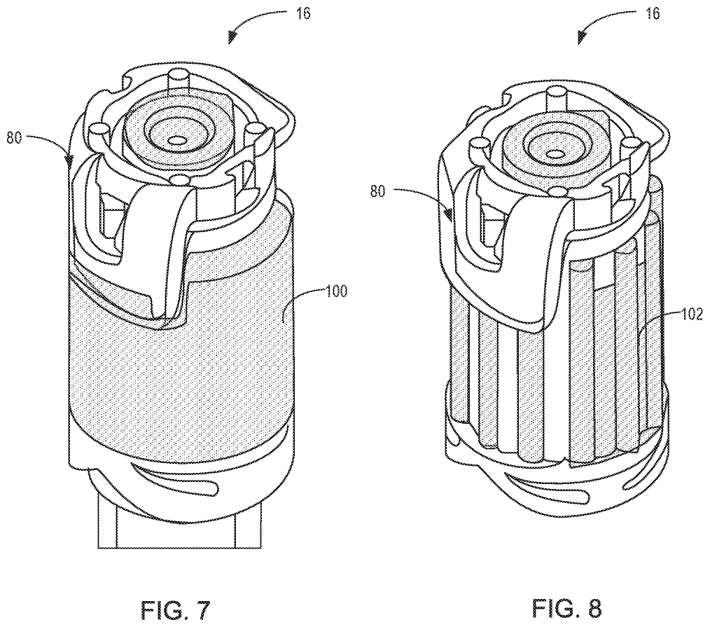

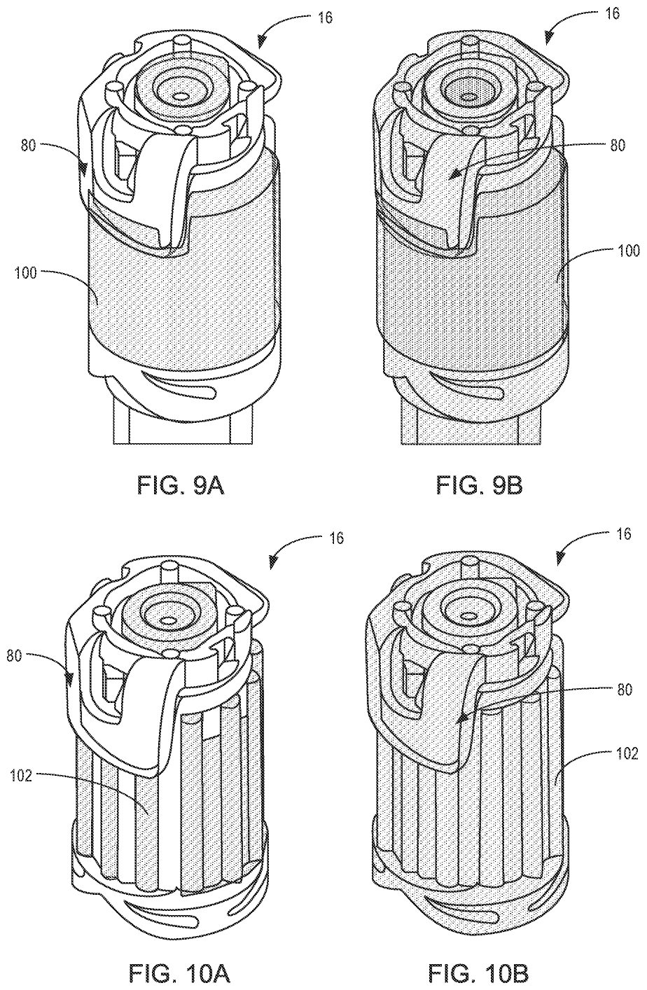

With reference to FIGS. 9A and 9B, according to one non-limiting aspect or example, the collection container 16 may include an additional or alternative feature for assisting in identifying the collection container 16 for a vision system for a pick-and-place automated machine or device. In some aspects, the vision system may be operated to determine a specific color of a cap of the collection container 16 to ensure the desired collection container 16 is being gripped and/or picked up. This cap color determination is a critical front-end step for successful automation of the pick-and-place machine or device. To assist in ensuring a uniform color is presented to the vision system for optical recognition of the collection container type, the cover 100 of the collection container 16 may be formed or manufactured with a colorant to match the surrounding portions of the collection container 16. In some examples, a uniform color distribution is not achieved with the top portion of the collection container 16 due to the various protruding and angled features of the collection container 16. Therefore, by using colorant for the cover 100, a larger uniform area is presented with the desired colorant to ensure the vision system can optically recognize the colorant in the collection container 16. In one example, the colorant could be molded in-color with the existing cap body of the collection container 16 or over a similarly colored cap body. In one example, shown in FIG. 9B, the entire cover 100 and the entire cap body of the collection container 16 may be made with the same colorant. In one example, the cover 100 and the cap body may be made of a yellow colorant. The yellow colorant could be a solid yellow or different shades or levels of translucency with a yellow color. It is also contemplated that colors other than yellow may be used with the cover 100 and the cap body. As shown in FIGS. 10A and 10B, according to one non-limiting embodiment or aspect of the present disclosure, the axial ribs 102 of the collection container 16 may also include a colorant, such as a yellow colorant, to provide a uniform color distribution across the cap body of the collection container 16. The color of the axial ribs 102 could be matched to the color of the surrounding cap body.

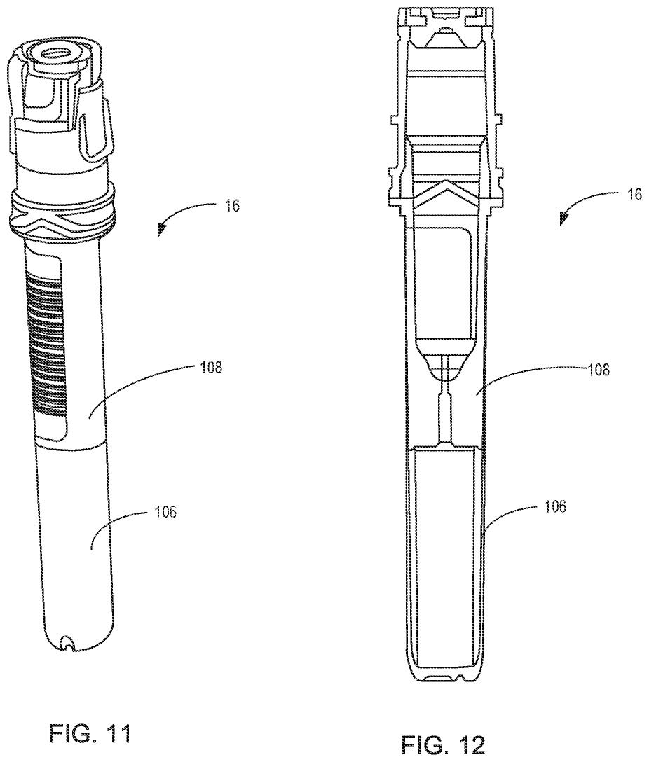

With reference to FIGS. 11 and 12, according to one non-limiting aspect or example of the present disclosure, an additional feature may be included on the collection container 16 to assist in interacting with a pick-and-place machine or device. In some pick-and-place machines or devices, an overall tube height for a collection container 16 must be achieved to properly mate with and interact with the pick-and-place machine or device. Further, some pick-and-place machines or devices need a single-wall of plastic on the collection container 16 to accurately assess the liquid level and sample volume in the collection container 16. As shown in FIGS. 11 and 12, the collection container 16 can be adapted to augment the tube overall height while only presenting a single wall of plastic for liquid level sensing in the pick-and-place machine or device. In one example, an extended portion 106 that attaches to a bottom of the collection container 16 may be provided. The extended portion 106 may be an adapter that can connect to any pre-existing collection container. The extended portion 106 may have arms 108 that are used to positively connect with the bottom portion of the collection container 16. The arms 108 may be removably attached or integrally formed with the collection container 16. By only attaching the extended portion 106 to the bottom of the collection container 16, the single wall of plastic is maintained for the collection container 16 for the liquid level sensing. The connection between the extended portion 106 and the collection container 16 creates a uniform surface to give the appearance of a uniform “extended” collection container 16.

The extended portion 106 may be included at point-of-manufacture as a permanent feature for the collection container 16 or as an accessory that is attached post-collection and prior to sending for lab processing. The extended portion 106 may be secured to the collection container 16 with a friction fit connection, an annular snap fit connection, a threaded connection, a bayonet connection, or an ultrasonic welding connection. The collection container 16 can alternatively be augmented to create a longer “false bottom” that eliminates the need for an accessory extender component.

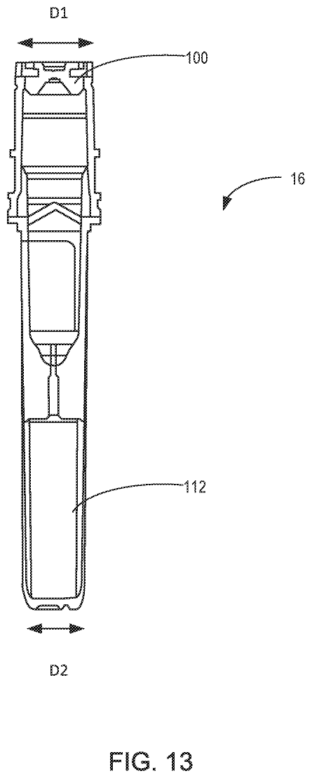

With reference to FIG. 13, according to one non-limiting aspect or example of the present disclosure, an additional feature may be included on the collection container 16 to assist in interacting with a pick-and-place machine or device. In some pick-and-place machines or devices, the collection container 16 must have a cap with a larger diameter than a bottom portion of the collection container 16 to properly mate with and interact with the pick-and-place machine or device. Due to the cap of the collection container 16 having a larger diameter than the tube of the collection container 16, the automation process of the pick-and-place machine or device is improved for determining tube orientation in the pick-and-place machine or device and automatically sorts for the correct orientation. Collection containers 16 that do not include this diameter difference do not allow for automated differentiation as the assembly has a uniform outer diameter. As shown in FIG. 13, according to one example, the collection container 16 may include a cap 110 that has a diameter D1 that is greater than a diameter D2 of the tube 112 of the collection container 16. The difference in diameter allows for orientation sorting in an automated workflow with the pick-and-place machine or device. The difference in diameter allows the pick-and-place machine or device to distinguish between the top of the collection container 16 (the cap 110) and the bottom of the collection container 16 (the tube 112).

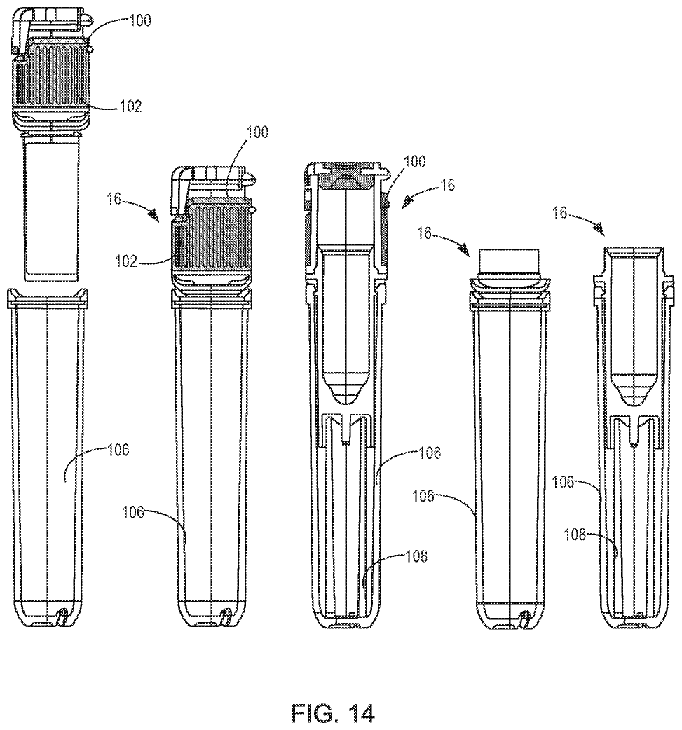

With reference to FIG. 14, according to a non-limiting embodiment or aspect of the present disclosure, a collection container 16 with a combination of a cover 100 with a plurality of axial ribs 102 is shown and described in detail. In some pick-and-place machines or devices, an overall tube height for a collection container 16 must be achieved to properly mate with and interact with the pick-and-place machine or device. Further, some pick-and-place machines or devices need a single-wall of plastic on the collection container 16 to accurately assess the liquid level and sample volume in the collection container 16. As shown in FIG. 14, the collection container 16 can be adapted to augment the tube overall height while only presenting a single wall of plastic for liquid level sensing in the pick-and-place machine or device. In one example, an extended portion 106 that attaches to a bottom of the collection container 16 may be provided. The extended portion 106 may be an adapter that can connect to any pre-existing collection container. The extended portion 106 may have arms 108 that are used to positively connect with the bottom portion of the collection container 16. The arms 108 may be removably attached or integrally formed with the collection container 16. By only attaching the extended portion 106 to the bottom of the collection container 16, the single wall of plastic is maintained for the collection container 16 for the liquid level sensing. The connection between the extended portion 106 and the collection container 16 creates a uniform surface to give the appearance of a uniform “extended” collection container 16.

The collection container 16 shown in FIG. 14 may include a cover member, such as cover 100, that is provided around an outer circumference of the collection container 16 to create a uniform radial surface for gripping and/or holding the collection container 16 during pick-and-place automation. The cover 100 may extend around the entire outer circumference of a top portion of the collection container 16. In one example, the cover 100 may be an over-mold of a rigid plastic or a semi-rigid elastomeric material. The cover 100 is configured to improve the ability of the arms or grippers of an automated machine or device to grip the collection container 16 for transport or relocation. The cover 100 may be formed as a single monolithic piece or as a plurality of pieces that are connected to one another. The cover may be opaque in color to assist in tube recognition. The cover may also provide uniform axial symmetry about a circumferential perimeter of the exterior surface of the at least a portion of the collection container 16, such as adjacent the open end.

In addition to the cover 100, the collection container 16 may also include a plurality of axial ribs 102 may be positioned around the circumference of the top portion of the collection container 16 to create a uniform radial surface for gripping and/or holding the collection container 16 during pick-and-place automation. The plurality of axial ribs 102 may extend around the entire outer circumference of the collection container 16. A longitudinal axis of each axial rib 102 may extend parallel to a longitudinal axis of the collection container 16. The axial ribs 102 ensure that there are not one or two protruding edges on the collection container 16 but, instead, includes a more uniform smooth surface. The axial ribs 102 are configured to fill in the negative space below the various protrusions on the collection container 16, including the negative space below the lid latch and the living hinge. The axial ribs 102 may be made of a rigid plastic or a semi-rigid elastomeric material. The axial ribs 102 may be provided on the collection container 16 during primary molding of the collection container 16 or as an over-molded feature on the collection container 16. In another example, instead of the axial ribs 102, a plurality of rings could be positioned along the length of the top portion of the collection container 16 to also create a more uniform smooth surface that can more easily be gripped by arms or grippers of an automated pick-and-place machine or device. The rings could be radial rings that extend around the outer circumference of the top portion of the collection container 16.

It is also contemplated that the cover 100 and axial ribs 102 illustrated in FIG. 14 may include the color differentiation illustrated and discussed in connection with FIGS. 9A-10B.

While embodiments of a capillary blood collection device are shown in the accompanying figures and described hereinabove in detail, other embodiments will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A collection container for holding a blood sample, the collection container comprising:
a tube having an open end, a closed bottom end, and a sidewall extending therebetween that defines a collection cavity, the sidewall defining an exterior side surface;
a lid removably engageable with the open end of the tube; and
a cover member for covering at least a portion of the exterior side surface of the tube proximate the lid,
wherein the cover member is configured to circumferentially cover at least a portion of the exterior side surface of the collection container to create a uniform smooth surface around a circumference of the collection container.

2. The collection container of claim 1, wherein the lid includes a release tab that releases the collection container from a blood collector attachment when pressed.

3. The collection container of claim 1, wherein the lid is connected to the collection container via a living hinge.

4. The collection container of claim 1, wherein the cover member is integrally formed with the collection container.

5. The collection container of claim 1, wherein the cover member includes a colorant to identify the cover member.

6. The collection container of claim 5, wherein the colorant is opaque and/or translucent.

7. The collection container of claim 1, wherein a diameter of the lid is greater than a diameter of the tube.

8. The collection container of claim 1, further comprising an extended portion removably attached to a bottom end of the tube.

9. A collection container for holding a blood sample, the collection container comprising:
a tube having an open end, a closed bottom end, and a sidewall extending therebetween that defines a collection cavity, the sidewall defining an exterior side surface;
a lid removably engageable with the open end of the tube; and
a plurality of ribs configured to cover at least a portion of the exterior side surface of the tube proximate the lid,
wherein the plurality of ribs are configured to circumferentially cover at least a portion of the exterior side surface to create a more uniform banded smooth surface around a circumference of the collection container.

10. The collection container of claim 9, wherein the plurality of ribs are disposed axially.

11. The collection container of claim 9, wherein the lid includes a release tab that releases the collection container from a blood collector attachment when pressed.

12. The collection container of claim 9, wherein the lid is connected to the collection container via a living hinge.

13. The collection container of claim 9, wherein the plurality of ribs is integrally formed with the collection container.

14. The collection container of claim 9, wherein the plurality of ribs includes a colorant to identify the plurality of ribs.

15. The collection container of claim 14, wherein the colorant is opaque and/or translucent.

16. The collection container of claim 9, wherein a diameter of the lid is greater than a diameter of the tube.

17. The collection container of claim 9, further comprising an extended portion removably attached to a bottom end of the tube.

18. A collection container for holding a blood sample, the collection container comprising:
a tube having an open end, a closed bottom end, and a sidewall extending therebetween that defines a collection cavity, the sidewall defining an exterior side surface;
a lid removably engageable with the open end of the tube; and
an extended portion removably engageable with a portion of the tube, such that the extended portion extends over the closed bottom end and is configured to increase an overall length of the tube.

19. The collection container of claim 18, wherein the extended portion is removably connected to the tube via a friction fit, an annular snap fit connection, a threaded connection, a bayonet connection, or an ultrasonic welding connection.

20. A collection container for holding a blood sample, the collection container comprising:
a tube having an open end, a closed bottom end, and a sidewall extending therebetween that defines a collection cavity, the sidewall defining an exterior side surface;
a lid removably engageable with the open end of the tube;
a cover member for covering at least a portion of the exterior side surface of the tube proximate the lid; and
a plurality of ribs positioned on the cover member and configured to cover at least a portion of the cover,
wherein the cover and the plurality of ribs are configured to circumferentially cover at least a portion of the exterior side surface to create a more uniform banded smooth surface around a circumference of the collection container.
